(19) United States
(12) Patent Application Publication
(10) Pub. No.: US 2026/0308670 A1
(43) Pub. Date: Oct. 8, 2026

(54) METHODS FOR ISOLATING A MICROBIAL ANALYTE

(71) Applicant: Gen-Probe Incorporated, San Diego, CA (US)

(72) Inventors: Anne NIINIVAARA, Vantaa (FI); Juha SAHARINEN, Espoo (FI)

(21) Appl. No.: 19/490,286

(22) PCT Filed: Jun. 7, 2024
(86) PCT No.: PCT/FI2024/050300

§ 371 Date: Dec. 4, 2025

(30) Foreign Application Priority Data
Jun. 12, 2023 (FI) 20235652

(51) Int. Cl. C12Q 1/6844; C12N 1/063; C12N 1/066; C12N 15/10; C12Q 1/6834; C12Q 1/6876

(52) U.S. Cl. CPC C12Q 1/6844; C12N 1/063; C12N 1/066; C12N 15/1017; C12Q 1/6834; C12Q 1/6876

(57) ABSTRACT

Disclosed are methods for selectively isolating a microbial cell analyte (e.g., a nucleic acid) from a sample containing or suspected of containing mammalian cells. The selective isolation method includes selective lysis of the mammalian cells and filtration of the resulting lysate through a filter that retains intact microbial cells, followed by release of the retained microbial cells from the filter. The microbial cells are then collected from the filter using reverse-flow elution or aspiration, and the collected microbial cells are lysed to release the analyte from the intact cells. The isolated analytes may be analyzed using a suitable assay depending on the type of analyte molecule.

### BACKGROUND

Many molecular diagnostics applications require the best possible sensitivity for being state-of the-art and competitive with products from other molecular diagnostics manufacturers. High sensitivity is a definite asset of typically all molecular diagnostics applications and a feature typically evaluated by the end-user.

In addition, certain cases pose sensitivity requirements that cannot be achieved by any typical sample extraction method, regardless of their sensitivity, due to low number of analyte molecules in the volume of the sample used for extraction. Some applications in this area include direct blood sepsis pathogen detection, liquid biopsies for circulating tumor cells (CTCs), circulating tumor DNA and exosomes (from, e.g., blood/plasma and urine matrices) as well as, e.g., water analytics and airborne pathogen detection.

For example, direct blood sample microbe detection would have immense value due to faster overall result of the pathogen presence, identification, and, e.g., antibiotics resistance properties. The current gold standard method is based on pathogen enrichment by blood culturing, which is both time consuming (e.g., 24-72 hours) and with low detection sensitivity even after culturing. Worldwide, approximately 30 million sepsis cases emerge annually, leading to 7-9 million deaths (one death every 3.5 seconds). The published data indicate an 8% decrease in survival rate for every hour of delayed antibiotics administration in sepsis, and that 15-40% of the sepsis patients receive inappropriate empirical antibiotics treatment due to inability to detect the causative pathogen promptly, underlining the importance of early pathogen detection, identification, and antibiotics resistance profiling.

In the field of direct blood sepsis testing, nucleic acid-based assays utilizing pathogen enrichment from the blood sample have been developed by Qvell, T2 Biosystems, and DNAe. All these assays are based on multiplex PCR, preceded by pathogen enrichment from the blood sample. The DNAe assay uses magnetic particles coupled with pathogen-specific antibodies, while the T2 Biosystems and Qvell assays use centrifugation for enrichment. The use of biomolecules such as antibodies for sample enrichment poses issues with storage/stability, lot-to-lot variation, and cost in addition to the incubation time. The use of centrifugation in a closed system results in bulky instruments and specific fluidics capable of retaining the correct fraction of the centrifugate.

European Patent No. EP2510123 discloses a method and a device employing selective lysis for isolating a microbial cell analyte from a sample, the method comprising the steps of providing a sample with eukaryotic cells, in particular animal cells, containing or suspected to contain a micro-organism, adding a nonionic detergent and a buffer to the sample to obtain a solution with a pH of about 9.5 or more, and incubating the solution for a time period sufficiently long to lyse the animal cells, filtering the first lysate through a filter having a pore size that retains the intact microbial cells, wherein the pore size is from about 0.1 μm to about 1 μm, and lysing the microbial cells to obtain a second lysate comprising an analyte. In EP2510123, it is further disclosed that the filter retaining the intact microbial cells is incubated with a lysis buffer comprising 200 mM sodium hydroxide (NaOH) and 0.5% SDS at 95° C. for 10 min.

There is a need for sample enrichment methods in various application areas that enable, for example, analyte detection sensitivity beyond what is achievable by conventional methods, as well as increased efficiency of cost, assay time, and instrumentation space.

### SUMMARY

Methods described herein are based on using selective filtering, combined with reverse-flow filter content collection and downstream off-filter lysis of microbial cells, to enrich a microbial cell analyte from a larger volume of a sample containing mammalian cells. In this manner, overall assay sensitivity can be improved by processing a larger sample volume. In addition, as microbes may vary with respect to their tolerances to lysis treatments, releasing and collecting microbial cells from the filter enable the use of stronger lysis methods that are not possible or are very challenging to conduct inside the filter. The concept is applicable to molecular diagnostics with downstream nucleic acid (e.g., RNA/DNA) amplification and detection by qPCR and/or microarray, but also for other detection modalities, including next-generation sequencing as well as for protein/other biomolecule detection by for example ELISA, protein arrays, MALDI-TOF, nanoLC/UPLC-ESI-MS, and other relevant amplification/separation/detection modalities.

A method for selectively isolating a microbial cell analyte from a sample in accordance with the present disclosure generally includes, (a) providing a sample containing mammalian cells and potentially containing microbial cells; (b) mixing the sample with a first solution that selectively lyses the mammalian cells to obtain a first lysate containing lysed mammalian cells and intact microbial cells, if present; (c) filtering the first lysate through a filter having a pore size that retains the intact microbial cells; (d) contacting the filter containing the retained microbial cells with a second solution that is effective to allow release of the microbial cells from the filter; (e) incubating the filter with the second solution under conditions that facilitate the release of the microbial cells from the filter; (f) collecting the microbial cells from the filter, wherein the collection step comprises (i) using an elution buffer to elute the microbial cells from the filter, wherein the direction of fluid flow through the filter is opposite the direction of fluid flow applied in the filtering step (c), or (ii) aspirating the microbial cells from the filter, wherein the microbial cells are aspirated from the same side of the filter initially contacted by the first lysate at step (c); and (g) lysing the microbial cells collected at step (f) to obtain a second lysate comprising an analyte released from the microbial cells, thereby selectively isolating a microbial cell analyte from the sample. In some embodiments, the analyte to be isolated is a nucleic acid. In other, non-mutually exclusive embodiments, the microbial cells are bacterial cells or yeast cells. In other, non-mutually exclusive variations, the mammalian cells are blood cells. In yet other non-mutually exclusive embodiments, the mammalian cells are human cells.

Particularly suitable filters for use in the method include filters comprising polyethersulfone (PES), cellulose, nylon, polyvinylidene fluoride (Poly(1,1-difluoroethylene), PVDF), polycarbonate, or glass (e.g., borosilicate glass) fiber. In certain variations, the filter has a pore size of about 1 μm or less (e.g., a pore size of from about 0.1 μm to about 1 μm, a pore size of from about 0.2 to about 1 μm, or a pore size of about 0.22 μm). In other, non-mutually exclusive variations, the filter comprises an asymmetric structure. In certain variations, pores on a first side of the asymmetric filter have a size from about 5 μm to about 20 μm and pores on a second side of the asymmetric filter have a size of up to about 0.2 μm.

In some embodiments of a method as above, the first solution comprises a chaotropic salt and a detergent. In some such embodiments, the chaotropic salt is guanidine hydrochloride and/or the detergent is saponin. In certain variations, the first solution further includes a second detergent such as, e.g., polysorbate 20.

In some embodiments of a method as above, the first solution comprises guanidine hydrochloride, polysorbate 20, saponin, and a buffer. In some such embodiments, guanidine hydrochloride is present in the first solution at a concentration of from about 1 M to about 8 M; polysorbate 20 is present in the first lysis solution at a concentration of from 1% (v/v) to about 10% (v/v); saponin is present in the first solution at a concentration of from about 1% (w/v) to about 10% (w/v); and/or the buffer in the first solution is Tris and is present at a concentration of from about 20 mM to about 200 mM. In more specific variations, guanidine hydrochloride is present in the first solution at a concentration of about 4 M; polysorbate 20 is present in the first solution at a concentration of 4% (v/v); saponin is present in the first solution at a concentration of about 4% (w/v); and/or Tris is present in the first solution at a concentration of from about 35 mM to about 45 mM (e.g., about 40.5 mM).

In certain embodiments of a method as above, the ratio of the first solution to the sample during the mixing step (b) is about 1:1. In other, non-mutually exclusive variations, the concentration of guanidine hydrochloride in the first lysate is from about 0.5 M to about 4 M; the concentration of polysorbate 20 in the first lysate is from about 0.5% (v/v) to about 5% (v/v); the concentration of saponin in the first lysate is from about 0.5% (w/v) to about 5% (w/v); and/or the buffer in the first lysis solution is Tris and the concentration of Tris in the first lysate is from about 10 mM to about 100 mM. In some such variations, the concentration of guanidine hydrochloride in the first lysate is about 2 M; the concentration of polysorbate 20 in the first lysate is about 2% (v/v); the concentration of saponin in the first lysate is about 2% (w/v); and/or the buffer in the first solution is Tris and the concentration of Tris in the first lysate is from about 18 mM to about 23 mM (e.g., about 20.25 mM).

In some embodiments of a method as above, the second solution comprises sodium hydroxide, dimethyl sulfoxide (DMSO), glycerol, a chelating agent, and a buffer. In some such embodiments, sodium hydroxide is present in the second solution at a concentration of from about 10 mM to about 100 mM; DMSO is present in the second solution at a concentration of from about 2% (v/v) to about 20% (v/v); glycerol is present in the second solution at a concentration of from about 1% (v/v) to about 15% (v/v); the chelating agent in the second lysis solution is 2,2′,2″,2′″-(Ethane-1,2-diyldinitrilo)tetraacetic acid (EDTA) and is present at a concentration of from about 0.1 mM to about 4 mM; and/or the buffer in the second solution is Tris and is present at a concentration of from about 1 mM to about 50 mM. In more specific variations, sodium hydroxide is present in the second solution at a concentration of about 30 mM; DMSO is present in the second solution at a concentration of about 10% (v/v); glycerol is present in the second solution at a concentration of about 7% (v/v); the chelating agent in the second lysis solution is EDTA and is present at a concentration of about 1 mM; and/or the buffer in the second solution is Tris and is present at a concentration of from about 5 mM to about 10 mM (e.g., about 7.5 mM).

In certain variations of a method as above, the conditions of step (e) comprise incubating the filter with the second solution at a temperature of from about 70° C. to about 99° C. (e.g., a temperature of from about 90° C. about 95° C.).

In certain embodiments, a method as above further includes (h) adding a neutralizing buffer to the eluate obtained in step (g). In some such embodiments, the neutralizing buffer may comprise hydrochloric acid (HCl) and Tris. Alternatively, in certain embodiments comprising analyzing the isolated target and wherein the analyzing steps include performing an amplification reaction (see infra), an amplification reaction mixture is buffered to neutralize the pH of the eluate/amplification reaction mixture (e.g., to make the pH of an eluate/amplification reaction mixture less alkaline).

In some embodiments, a method as above further includes a washing step between steps (c) and (d), wherein the washing step comprises flowing a wash buffer through the filter. In some such embodiments, the washing step is performed once after step (c) and before step (d). In other embodiments, the filtering step (c) and the wash step are performed in multiple, successive iterations before step (d), wherein each iteration of step (c) comprises filtering a portion of the first lysate through the filter. A particularly suitable wash buffer comprises EDTA and Tris; in some such variations, the wash buffer further comprises polysorbate 20, which may be present in the wash buffer at a concentration of from about 0.5% (v/v) to about 5% (v/v). In other embodiments, the wash buffer is the same as the second solution or a diluted concentration of the second lysis solution. In alternative variations, the method does not comprise a washing step between steps (c) and (d).

In some variations of a method as above wherein step (f) comprises (i) using the elution buffer to elute the microbial cells from the filter, the elution buffer comprises EDTA and Tris. In some such embodiments, the elution buffer further comprises polysorbate 20, which may be present in the elution buffer at a concentration of from about 0.5% (v/v) to about 5% (v/v).

In certain embodiments of a method as above, lysing the microbial cells at step (g) comprises physical lysis. Particularly suitable physical lysis methods include one of more of sonication, ultrasound, bead-beating, radiolysis, and electrolysis. In some variations, the physical lysis comprises electromagnetic lysis.

A method as above may further include analyzing the isolated analyte. For example, where the isolated analyte is a nucleic acid, steps for analyzing the isolated nucleic acid may include (i) performing an amplification reaction using the isolated nucleic acid as a template to generate an amplification product; and (ii) detecting the amplification product. In some such embodiments, the amplification reaction is PCR such as, e.g., quantitative PCR (qPCR). In other variations, the amplification is an isothermal amplification reaction such as, e.g., a transcription-mediated amplification reaction. The detection step (ii) may be performed in real time. The method may optionally include an analyte isolation/purification step for isolating a target analyte of interest from non-target analytes among the microbial analyte.

In some embodiments of a method as above and further comprising analyzing an isolated nucleic acid analyte, the analyzing step includes immobilizing the isolated nucleic acid or amplification product on a solid support. In some such embodiments, the isolated nucleic acid or amplification product is hybridized to an immobilized probe attached to the solid support such as, for example, an immobilized probe contained within a nucleic acid array.

Representative embodiments of the method are further set forth below.

### EMBODIMENTS

Embodiment 1. A method for selectively isolating a microbial cell analyte from a sample, the method comprising: (a) providing a sample containing mammalian cells and potentially containing microbial cells; (b) mixing the sample with a first solution that selectively lyses the mammalian cells to obtain a first lysate containing lysed mammalian cells and intact microbial cells, if present; (c) filtering the first lysate through a filter having a pore size that retains the intact microbial cells; (d) contacting the filter containing the retained microbial cells with a second solution that is effective to allow release of the microbial cells from the filter; (e) incubating the filter with the second solution under conditions that facilitate the release of the microbial cells from the filter; (f) collecting the microbial cells from the filter, wherein said collection step comprises (i) using an elution buffer to elute the microbial cells from the filter, wherein the direction of fluid flow through the filter is opposite the direction of fluid flow applied in the filtering step (c), or (ii) aspirating the microbial cells from the filter, wherein the microbial cells are aspirated from the same side of the filter initially contacted by the first lysate at step (c); and (g) lysing the microbial cells collected at step (f) to obtain a second lysate comprising an analyte released from the microbial cells, thereby selectively isolating a microbial cell analyte from the sample.

Embodiment 2. The method of Embodiment 1, wherein the microbial cells are bacterial cells.

Embodiment 3. The method of Embodiment 2, wherein the bacterial cells are Gram-positive bacterial cells.

Embodiment 4. The method of Embodiment 2, wherein the bacterial cells are Gram-negative bacterial cells.

Embodiment 5. The method of Embodiment 2, wherein the microbial cells are yeast cells.

Embodiment 6. The method of any one of Embodiments 1 to 5, wherein the filter has a pore size of about 1 μm or less.

Embodiment 7. The method of Embodiment 6, wherein the filter has a pore size of from about 0.1 μm to about 1 μm.

Embodiment 8. The method of Embodiment 7, wherein the filter has a pore size of about 0.22 μm.

Embodiment 9. The method of any one of Embodiments 1 to 8, wherein the filter comprises polyethersulfone (PES), cellulose, nylon, polyvinylidene fluoride (PVDF), polycarbonate, or glass fiber.

Embodiment 10. The method of any one of Embodiments 1 to 9, wherein the filter comprises an asymmetric structure, and, preferably, the asymmetric filter comprises pores on a first side having a size from about 5 μm to about 20 μm and pores on a second side having a size of up to about 0.2 μm.

Embodiment 11. The method of any one of Embodiments 1 to 10, wherein the first solution comprises a chaotropic salt and a detergent.

Embodiment 12. The method of Embodiment 11, wherein the chaotropic salt is guanidine hydrochloride.

Embodiment 13. The method of Embodiment 11 or 12, wherein the detergent is saponin.

Embodiment 14. The method of Embodiment 13, wherein the first solution further comprises a second detergent.

Embodiment 15. The method of Embodiment 14, wherein the second detergent is polysorbate 20.

Embodiment 16. The method of any one of Embodiments 1 to 10, wherein the first solution comprises guanidine hydrochloride, polysorbate 20, saponin, and a buffer.

Embodiment 17. The method of Embodiment 16, wherein guanidine hydrochloride is present in the first solution at a concentration of from about 1 M to about 8 M.

Embodiment 18. The method of Embodiment 17, wherein guanidine hydrochloride is present in the first solution at a concentration of about 4 M.

Embodiment 19. The method of any one of Embodiments 16 to 18, wherein polysorbate 20 is present in the first solution at a concentration of from about 10% (v/v) to about 10% (v/v).

Embodiment 20. The method of Embodiment 19, wherein polysorbate 20 is present in the first solution at a concentration of 4% (v/v).

Embodiment 21. The method of any one of Embodiments 16 to 20, wherein saponin is present in the first solution at a concentration of from about 1% (w/v) to about 10% (w/v).

Embodiment 22. The method of any Embodiment 21, wherein saponin is present in the first solution at a concentration of about 4% (w/v).

Embodiment 23. The method of any one of Embodiments 16 to 22, wherein the buffer in the first solution is Tris and is present at a concentration of from about 20 mM to about 200 mM.

Embodiment 24. The method of Embodiment 23, wherein Tris is present in the first solution at a concentration of from about 35 mM to about 45 mM.

Embodiment 25. The method of any one of Embodiments 1 to 24, wherein during the mixing step (b) the ratio of the first solution to the sample is about 1:1.

Embodiment 26. The method of Embodiment 16, wherein the concentration of guanidine hydrochloride in the first lysate is from about 0.5 M to about 4 M.

Embodiment 27. The method of Embodiment 26, wherein the concentration of guanidine hydrochloride in the first lysate is about 2 M.

Embodiment 28. The method of any one of Embodiments 16, 26, and 27, wherein the concentration of polysorbate 20 in the first lysate is from about 0.5% (v/v) to about 5% (v/v).

Embodiment 29. The method of Embodiment 28, wherein the concentration of polysorbate 20 in the first lysate is about 2% (v/v).

Embodiment 30. The method of any one of Embodiments 16 and 26 to 29, wherein the concentration of saponin in the first lysate is from about 0.5% (w/v) to about 5% (w/v).

Embodiment 31. The method of Embodiment 30, wherein the concentration of saponin in the first lysate is about 2% (w/v).

Embodiment 32. The method of any one of Embodiments 16 and 26 to 31, wherein the buffer in the first solution is Tris and the concentration of Tris in the first lysate is from about 10 mM to about 100 mM.

Embodiment 33. The method of Embodiment 32, wherein the concentration of Tris in the first lysate is from about 18 mM to about 23 mM.

Embodiment 34. The method of any one of Embodiments 1 to 33, wherein the second solution comprises sodium hydroxide, dimethyl sulfoxide (DMSO), glycerol, a chelating agent, and a buffer.

Embodiment 35. The method of Embodiment 34, wherein sodium hydroxide is present in the second solution at a concentration of from about 10 mM to about 100 mM.

Embodiment 36. The method of Embodiment 35, wherein sodium hydroxide is present in the second solution at a concentration of about 30 mM.

Embodiment 37. The method of any one of Embodiments 34 to 36, wherein DMSO is present in the second solution at a concentration of from about 2% (v/v) to about 20% (v/v).

Embodiment 38. The method of Embodiment 37, wherein DMSO is present in the second solution at a concentration of about 10% (v/v).

Embodiment 39. The method of any one of Embodiments 34 to 38, wherein glycerol is present in the second solution at a concentration of from about 1% (v/v) to about 15% (v/v).

Embodiment 40. The method of Embodiment 39, wherein glycerol is present in the second solution at a concentration of about 7% (v/v).

Embodiment 41. The method of any one of Embodiments 34 to 40, wherein the chelating agent in the second solution is 2,2′,2″,2′″-(Ethane-1,2-diyldinitrilo)tetraacetic acid (EDTA) and is present at a concentration of from about 0.1 mM to about 4 mM.

Embodiment 42. The method of Embodiment 41, wherein EDTA is present in the second solution at a concentration of about 1 mM.

Embodiment 43. The method of any one of Embodiments 34 to 42, wherein the buffer in the second solution is Tris and is present at a concentration of from about 1 mM to about 50 mM.

Embodiment 44. The method of Embodiment 43, wherein Tris is present in the second solution at a concentration of from about 5 mM to about 10 mM.

Embodiment 45. The method of any one of Embodiments 1 to 44, wherein the conditions that facilitate the release of the microbial cells from the filter at step (e) comprise incubating the filter with the second solution at an incubation temperature of from about 70° C. to about 99° C.

Embodiment 46. The method of Embodiment 45, wherein the incubation temperature is from about 90° C. to about 95° C.

Embodiment 47. The method of any one of Embodiments 1 to 46, further comprising a washing step between steps (c) and (d), wherein the washing step comprises flowing a wash buffer through the filter.

Embodiment 48. The method of Embodiment 47, wherein the filtering step (c) and the wash step are performed in multiple, successive iterations before step (d), wherein each iteration of step (c) comprises filtering a portion of the first lysate through the filter.

Embodiment 49. The method of Embodiment 47 or 48, wherein the wash buffer comprises EDTA and Tris.

Embodiment 50. The method of Embodiment 49, wherein the wash buffer further comprises polysorbate 20.

Embodiment 51. The method of Embodiment 50, wherein the polysorbate 20 is present in the wash buffer at a concentration of from about 0.5% (v/v) to about 5% (v/v).

Embodiment 52. The method of any one of Embodiments 1 to 51, wherein step (f) comprises (i) using the elution buffer to elute the microbial cells from the filter, wherein the direction of fluid flow through the filter is opposite the direction of fluid flow applied in the filtering step (c).

Embodiment 53. The method of Embodiment 52, wherein the elution buffer comprises EDTA and Tris.

Embodiment 54. The method of Embodiment 53, wherein the elution buffer further comprises polysorbate 20.

Embodiment 55. The method of Embodiment 54, wherein the polysorbate 20 is present in the elution buffer at a concentration of from about 0.5% (v/v) to about 5% (v/v).

Embodiment 56. The method of any one of Embodiments 1 to 51, wherein step (f) comprises (ii) aspirating the microbial cells from the filter, wherein the microbial cells are aspirated from the same side of the filter initially contacted by the first lysate at step (c).

Embodiment 57. The method of any one of Embodiments 1 to 56, wherein lysing the microbial cells at step (g) comprises physical lysis.

Embodiment 58. The method of Embodiment 57, wherein the physical lysis comprises sonication, ultrasound, bead-beating, radiolysis, and/or electrolysis.

Embodiment 59. The method of Embodiment 57 or 58, wherein the physical lysis comprises electromagnetic lysis.

Embodiment 60. The method of any one of Embodiments 1 to 59, further comprising (h) adding a neutralizing buffer to the second lysate obtained in step (g).

Embodiment 61. The method of Embodiment 60, wherein the neutralizing buffer comprises hydrochloric acid (HCl) and Tris.

Embodiment 62. The method of any one of Embodiments 1 to 61, wherein the mammalian cells are human cells.

Embodiment 63. The method of any one of Embodiments 1 to 62, wherein the mammalian cells are blood cells.

Embodiment 64. The method of any one of Embodiments 1 to 63, wherein the analyte is a nucleic acid.

Embodiment 65. The method of any one of Embodiments 1 to 64, further comprising analyzing the isolated analyte.

Embodiment 66. The method of Embodiment 64, further comprising analyzing the isolated nucleic acid.

Embodiment 67. The method of Embodiment 66, wherein analyzing the isolated nucleic acid comprises: (i) performing an amplification reaction using the isolated nucleic acid as a template to generate an amplification product; and (ii) detecting the amplification product.

Embodiment 68. The method of Embodiment 67, wherein the amplification reaction is PCR.

Embodiment 69. The method of Embodiment 67, wherein the amplification reaction is an isothermal amplification reaction.

Embodiment 70. The method of Embodiment 69, wherein the isothermal amplification reaction is a transcription-mediated amplification reaction.

Embodiment 71. The method of any one of Embodiments 67 to 70, wherein the detecting step (ii) is performed in real time.

Embodiment 72. The method of any one of Embodiments 67 to 70, wherein analyzing the isolated nucleic acid comprises immobilizing the isolated nucleic acid or amplification product on a solid support.

Embodiment 73. The method of Embodiment 72, wherein the isolated nucleic acid or amplification product is hybridized to an immobilized probe attached to the solid support.

Embodiment 74. The method of Embodiment 73, wherein the immobilized probe is contained within a nucleic acid array.

These and other aspects of the invention will become evident upon reference to the following detailed description.

### Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise.

The terms “microbe,” “microorganism,” and “microbial,” as used herein, refer to bacteria, archaea, fungi, and protists. In some embodiments, a microbe is a prokaryote (i.e., a species of bacteria or archaea). In other embodiments, a microbe is a microbial species having a cell wall such as any species of bacteria, archaea, or fungi, or some protist species.

The term “intact,” as used herein in reference to a microbial cell, means a cell with a substantially non-disrupted cell membrane and/or cell wall, such that intracellular constituents are generally retained within the cell.

The term “release,” as used herein in reference to microbial cells or other content captured on a filter, means the detachment of the captured content from the membrane. Release of captured filter content may include partial lysis of microbial cells. For example, in certain embodiments, a release process (e.g., chemical incubation, with or without heating) may lyse certain types of microbes, but tough-to-lyse Gram-positive microbes and yeasts will remain intact.

An “analyte,” as used herein, refers to a substance or one or more constituents thereof that is for identification and/or characterization, such as, e.g., detection via a probe or sequencing. Examples of analytes include, without limitation, DNA, RNA, and protein. In the context of the present disclosure, analytes are constituents of microbial cells.

“Sample” includes any specimen that may contain an analyte. Samples include “biological samples,” which include any tissue or material derived from a living or dead mammal (e.g., human). Also, samples may include processed samples, such as, e.g., those obtained from passing samples over or through a filtering device, or following centrifugation, or by adherence to a medium, matrix, or support.

A “detergent” refers to a substance that can disperse a hydrophobic substance (e.g., lipids) in water by emulsification and which can be used to lyse or solubilize a biological sample for subsequent analysis. Detergents may be ionic or nonionic.

A buffer refers to a weak acid or weak base used to maintain the pH of a solution.

The term “asymmetric structure,” as used herein in reference to a filter, refers to a filter with pores having variable pore diameters and wherein pores on one side of the filter are generally larger than pores on an opposite side of the filter. In some embodiments, an asymmetric structure comprises a single filter membrane. In other variations, an asymmetric structure comprise multiple filter membranes wherein each filter membrane has a different pore size (e.g., a first filter membrane having a larger pore size as a prefiltering membrane, and then one more additional membranes, wherein the additional membrane most distal to the first membrane has the smallest pore size). Unless the context clearly dictates otherwise, reference to “a membrane” or “the membrane” includes reference to multiple membranes that may be within a filter, such as a filter with an asymmetric structure.

A “nucleic acid” refers to a multimeric compound comprising nucleotides or analogs that have nitrogenous heterocyclic bases or base analogs linked together to form a polymer, including conventional RNA, DNA, mixed RNA-DNA, and analogs thereof.

The term “nucleic acid array,” as used herein, refers to a solid support upon which a collection of target-specific nucleic acids has been placed at defined locations, either by spotting or direct synthesis.

A “nucleotide” as used herein is a subunit of a nucleic acid consisting of a phosphate group, a 5-carbon sugar, and a nitrogenous base (also referred to herein as “nucleobase”). The 5-carbon sugar found in RNA is ribose. In DNA, the 5-carbon sugar is 2′-deoxyribose.

“Oligomer,” “oligonucleotide,” or “oligo” refers to a nucleic acid of generally less than 1,000 nucleotides (nt), including those in a size range having a lower limit of about 5 nt and an upper limit of about 900 nt. The term oligonucleotide does not denote any particular function to the reagent; rather, it is used generically to cover all such reagents described herein. Oligomers may be referred to by a functional name (e.g., capture probe, detection probe, primer, or promoter primer) but those skilled in the art will understand that such terms refer to oligomers.

“Pore size” or any reference to the size of a pore refers to a measure of each pore characterizing the size of the largest particle that will pass through the pore. For example, in the context of a pore having a circular or generally circular transverse shape, the pore size may refer to the diameter of the shape. In the context of pores having a more rectangular or oval shape, the pore size may refer to the smaller width dimension of the shape, which corresponds to the width of the largest particle that may pass through the pore.

The term “target sequence” or “target nucleic acid sequence” as used herein refers to the particular nucleotide sequence of a nucleic acid analyte that is to be amplified and/or detected. The “target sequence” includes the complexing sequences to which oligonucleotides (e.g., priming oligonucleotides and/or promoter oligonucleotides) complex during an amplification processes (e.g., PCR, TMA). Unless the context clearly dictates otherwise, where the nucleic acid analyte is originally single-stranded, the term “target sequence” will also refer to the sequence complementary to the “target sequence” as present in the nucleic acid analyte, and where the nucleic acid analyte is originally double-stranded, the term “target sequence” refers to both the sense (+) and antisense (−) strands.

“Nucleic acid amplification” refers to any well-known in vitro procedure that produces multiple copies of a target nucleic acid sequence. Examples of such procedures include transcription-associated methods, e.g., transcription-mediated amplification (TMA), nucleic acid sequence-based amplification (NASBA) and others (e.g., U.S. Pat. Nos. 5,399,491, 5,554,516, 5,437,990, 5,130,238, 4,868,105, and 5,124,246), replicase-mediated amplification (e.g., U.S. Pat. No. 4,786,600), polymerase chain reaction (PCR) (e.g., U.S. Pat. Nos. 4,683,195, 4,683,202, and 4,800,159), ligase chain reaction (LCR) (e.g., EP Patent No. 0320308), and strand-displacement amplification (SDA) (e.g., U.S. Pat. No. 5,422,252).

By “amplicon” or “amplification product” is meant a nucleic acid molecule generated in a nucleic acid amplification reaction and which is derived from a nucleic acid analyte. An amplicon or amplification product contains a target nucleic acid sequence that may be of the same or opposite sense as the nucleic acid analyte.

An “amplification oligonucleotide” or “amplification oligomer” is an oligonucleotide that hybridizes to a nucleic acid analyte and participates in a nucleic acid amplification reaction, e.g., serving as a primer. Amplification oligomers can have 3′ ends that are extended by polymerization as part of the nucleic acid amplification reaction. Amplification oligomers can alternatively have 3′ ends that are not extended by polymerization, but provide a component that facilitates nucleic acid amplification, e.g., a promoter sequence joined 5′ to the target-specific sequence of the amplification oligomer. Such an amplification oligomer is referred to as a promoter provider. Amplification oligomers that provide both a 3′ target-specific sequence that is extendable by polymerization and a 5′ promoter sequence are referred to as promoter primers. Amplification oligomers may be optionally modified to include 5′ non-target-specific sequences such as tags, promoters (as mentioned), or other sequences used or useful for manipulating or amplifying the primer or target oligonucleotide.

“Detection probe oligomer,” “detection probe,” or “probe” refers to an oligomer that hybridizes specifically to a target sequence, including an amplified product, under conditions that promote nucleic acid hybridization, for detection of a nucleic acid analyte. Detection may either be direct (i.e., probe hybridized directly to the target) or indirect (i.e., a probe hybridized to an intermediate structure that links the probe to the target). A probe's target-specific sequence generally refers to the specific sequence within a larger sequence which the probe hybridizes specifically. A detection probe may include target-specific sequence(s) and non-target-specific sequence(s). Such non-target-specific sequences can include sequences which will confer a desired secondary or tertiary structure, such as a hairpin structure, which can be used to facilitate detection and/or amplification.

“Label” or “detectable label” refers to a moiety or compound joined directly or indirectly to a probe that is detected or leads to a detectable signal. Direct joining may use covalent bonds or non-covalent interactions (e.g., hydrogen bonding, hydrophobic or ionic interactions, and chelate or coordination complex formation) whereas indirect joining may use a bridging moiety or linker (e.g., via an antibody or additional oligonucleotide(s), which amplify a detectable signal). Any detectable moiety may be used, e.g., radionuclide, ligand such as biotin or avidin, enzyme, enzyme substrate, reactive group, chromophore such as a dye or particle (e.g., latex or metal bead) that imparts a detectable color, luminescent compound (e.g., bioluminescent, phosphorescent, or chemiluminescent compound such as an acridinium ester (“AE”) compound), and fluorescent compound (i.e., fluorophore). Fluorophores may be used in combination with a quencher molecule that absorbs light when in close proximity to the fluorophore to diminish background fluorescence. Detectably labeled probes include, for example, hydrolysis (e.g., TaqMan™) probes, AE-labeled probes, molecular torches, and molecular beacons.

Reference to a numerical range herein (e.g., “X to Y” or “from X to Y”) includes the endpoints defining the range and all values falling within the range.

Unless otherwise apparent from the context, when a value is expressed as “about” X or “approximately” X, the stated value of X will be understood to be accurate to ±10%. In specific reference to a temperature value, the term “about” or “approximately” means±1° C.

To the extent used herein, the terms “first” and “second” preceding the name of an element (e.g., a solution or a component thereof) are used for identification purposes to distinguish between similar elements, and are not intended to necessarily imply order, nor are the terms “first” and “second” intended to preclude the inclusion of additional similar elements. Furthermore, use of the term “first” preceding the name of an element does not necessarily imply or require that there be additional, e.g., “second,” “third,” etc., such element(s).

### Description

The present disclosure provides methods for selectively isolating a microbial cell analyte from a sample containing mammalian cells. The disclosed methods are particularly useful, e.g., for increasing sensitivity of downstream molecular diagnostic assays targeting the isolated analyte. For example, detection sensitivity in analytics applications such as, e.g., nucleic acid amplification and detection, is generally dependent on the amount of analyte introduced to a detection reaction. While typical molecular diagnostics applications are considered of high sensitivity, typical analyte extraction procedures for such applications (for example, DNA or RNA extraction for downstream use in amplification) do not provide the needed sensitivity. In certain aspects, the present disclosure addresses this problem by providing a method for enriching a microbial cell analyte, which may be present in relatively low amounts in a larger volume of a sample containing mammalian cells, using selective lysis to lyse mammalian cells, filtering the lysate to capture and isolate intact microbial cells (while mammalian cell constituents released by the selective lysis step will pass through the filter), releasing and collecting the captured filter content using reverse-flow elution or aspiration, and lysing the microbial cells collected from the filter. In this manner, overall assay sensitivity can be improved. For example, in addition to enrichment of the microbial cells achieved using selective lysis and filtering, the off-filter lysis of the microbial cells allows for a wider range of lysis methods since microbes have very different tolerances to different (e.g., chemical or enzymatic) lysis techniques, which can render on-filter lysis very challenging or even impossible. Using methods of the present disclosure, intact microbial cells containing an analyte are not necessarily lysed inside the filter but downstream of the collection step using an appropriate lysis treatment. The methods and corresponding systems are applicable, for example, to molecular diagnostics for nucleic acid detection (e.g., by quantitative PCR, nucleic acid array, and/or other microarray, and other nucleic acid detection modalities such as, for example, next generation sequencing) as well as detection of other biomolecules such as protein (e.g., by ELISA, protein arrays, MALDI-TOF, nanoLC/UPLC-ESI-MS, and other relevant detection modalities).

One concept of the disclosure is to use size selection filtering for enrichment of microbial organisms from a vast background matrix volume (such as, blood or other biological matrix), thus leading both to enrichment of the analyte concentration in the sample as well as replacing the sample matrix with one more compatible downstream analytics procedures (e.g., PCR or other detection methods, including immunological detection). Accordingly, a large sample volume can be processed for detecting analytes that may be present in very low concentrations. The size selection is achieved by using specific filters with selected pore sizes and filter material, optionally with asymmetric structure such that pore size decreases moving downstream through one or more filter membrane(s). In some examples, prokaryotic and eukaryotic target organism (bacteria and yeast) are trapped with filters having, e.g., 0.22 μm pore sizes and composed of, e.g., polyethersulfone (PES) or some other applicable membrane material. Selected membrane size and material allows efficient capture of the organisms containing the target analytes, and, moreover, the membrane type is not easily clogged even by using larger sample volume or by excessive washing of the membrane for particles smaller than the pore size.

To bring the analytes to the downstream analytics processes (such as, e.g., PCR, isothermal RNA/DNA amplification, protein or other biomolecule analyte detection), off-filter lysis is used. Microbial organisms captured by the filter are released from the capturing filter by chemical and/or physical methods, and the microbial cells are collected from the filter for downstream lysis. Collection of the microbial cells may employ reverse elution flow on the filter by flowing the sample solution in one direction through the filter membrane and flowing an elution reagent in the opposite direction through the filter membrane. Alternatively, the collection step comprises aspirating microbial cells from the filter, wherein the cells are collected from the same side of the filter initially contacted by the sample.

The reverse flow aspect of the present disclosure, combined with off filter lysis of collected microbes, is particularly advantageous for isolation of microbial cell analytes. For example, methods utilizing elution through the membrane following “in-filter-lysis” have more risks of not achieving efficient enrichment of microbial analytes than with lysis performed outside the filter as in the present methods. Also, while in-filter-lysis is possible for microbes that are more easily lysed—and with sonication also hard-to-lyse microbes can be broken and the analytes released—these methods are more difficult to design to an instrument. The reverse-flow method of the present disclosure can be readily implemented to an instrument and shows very good performance (see, e.g., Example 3, infra).

A method for selectively isolating a microbial cell analyte from a sample generally includes the following steps: (a) providing a sample containing or potentially containing mammalian cells and microbial cells; (b) mixing the sample with a first solution that selectively lyses the mammalian cells, if present, to obtain a first lysate containing lysed mammalian cells and intact microbial cells, if present; (c) filtering the first lysate through a filter having a pore size that retains the intact microbial cells but passes through the lysed mammalian cells; (d) contacting the filter containing the retained microbial cells with a second solution that is effective to allow release of the microbial cells from the filter (e) incubating the filter with the second solution under conditions that facilitate the release of the microbial cells from the filter; (f) collecting the microbial cells from the filter, wherein the collection step comprises (i) using an elution buffer to elute the microbial cells from the filter, wherein the direction of fluid flow through the filter is opposite the direction of fluid flow applied in the filtering step (c), or (ii) aspirating the microbial cells from the filter, wherein the microbial cells are aspirated from the same side of the filter initially contacted by the first lysate at step (c); and (g) lysing the microbial cells collected at step (f) to obtain a second lysate comprising an analyte released from the microbial cells, thereby selectively isolating a microbial cell analyte from the sample.

A range of samples containing or potentially containing mammalian cells and microbial cells may be analyzed in accordance with the present disclosure. In typical variations, a sample will be known to contain mammalian cells and will be at least suspected of containing one or more microorganisms. In some such variations, the sample has been isolated from a mammal. For example, particularly suitable samples for analysis using a method as disclosed herein are mammalian (e.g., human) blood samples for, e.g., blood sepsis pathogen detection. In other embodiments, a sample is not known to contain but is suspected of containing mammalian cells, such as a sample that is not isolated from a mammal but is derived from elsewhere (e.g., an environmental sample). The microbial cell can be any bacterial, archaeal, fungal, or protist species, particularly species characterized by a cell wall. Although in typical variations the microbial cell is a pathogenic microorganism or microorganism causing an infection in the body, the microbial cell may be pathogenic or non-pathogenic. In addition, the step of providing the sample need not encompass the isolation of the sample from a mammal or other source but may be performed by retrieving a sample from, e.g., a container where the sample is stored, or by initiating contact of the sample with the first lysis solution just prior to the mixing step (b).

Non-limiting examples of microbes that may be targeted for analyte isolation in accordance with the present disclosure include gram-negative bacteria such as, for example, Acinetobacter baumannii, Bacteroides fragilis, Enterobacterales (e.g., E. cloacae, E. coli, K aerogenes, K. oxytoca, K. pneumoniae, Proteus, Salmonella, S. marcescens), Haemophilus influenzae, Neisseria meningitidis, Pseudomanas aeruginosa, and Stenotrophomonas maltophilia; gram-positive bacteria such as, for example, Enterococcus faecalis, Enterococcus faecium, Listeria monocytogenes, Staphylococcus (e.g., S. aureus, S. epidermis, S. lugdunensis), and Streptococcus (e.g., S. agalactiae, S. pneumoniae, S. pyogenes); and yeast such as, for example, Candida albicans, Candida auris, Candida glabrata, Candida krusei, Candida parapsilosis, Candida tropicalis, Cryptococcus neogormans, and Cryptococcus gatti.

As previously indicated, the first solution is capable of lysing the mammalian cells while leaving any microbial cells, including microbial cells of interest, intact. The first solution may be mixed with the sample at any suitable ratio, based on the solution components and respective concentrations, to achieve effective concentrations of solution components in the final mixture for selective lysis. Exemplary ratios of first solution to sample include ratios of from about 3:1 to about 1:3, from about 2:1 to about 1:2, from about 1.5:1 to about 1:1.5, or about 1:1.

Typically, the first solution comprises a chaotropic salt and a detergent. A particularly suitable chaotropic salt is guanidine hydrochloride, which may be present in the first solution at, for example, a concentration of from about 1 M to about 8 M, from about 1 M to about 6 M, from about 1 M to about 5 M, from about 2 M to about 8 M, from about 2 M to about 6 M, from about 2 M to about 5 M, from about 3 M to about 8 M, from about 3 M to about 6 M, from about 3 M to about 5 M, or about 4 M. Suitable effective concentrations for guanidine hydrochloride (i.e., concentration in the first lysis reaction after mixing with the sample, or concentration in the final lysate) include, e.g., concentrations of from about 0.5 M to about 4 M, from about 0.5 M to about 3 M, from about 0.5 M to about 2.5 M, from about 1 M to about 4 M, from about 1 M to about 3 M, from about 1 M to about 2.5 M, from about 1.5 M to about 4 M, from about 1.5 M to about 3 M, from about 1.5 M to about 2.5 M, or about 2 M. Other suitable chaotropic salts include, e.g., guanidinium thiocyanate, urea, and lithium perchlorate.

A detergent for use in the first solution may be a non-ionic detergent, an ionic detergent, or a zwitterionic detergent. Mild detergents and/or low detergent concentrations are typically used in order to keep microbial cells intact. Non-ionic detergents such as, for example, saponin or a polyoxyethylene surfactant are preferred. Saponin is particularly preferred and may be present in the first lysis solution at, for example, a concentration of from about 1% (w/v) to about 10% (w/v), from about 2% (w/v) to about 8% (w/v), from about 2% (w/v) to about 6% (w/v), from about 3% (w/v) to about 5% (w/v), or about 4% (w/v); and saponin may be used at an effective concentration (i.e., concentration in the first lysis reaction after mixing with the sample, or concentration in the final lysate) of from about 0.5% (v/v) to about 5% (v/v), from about 1% (v/v) to about 4% (v/v), from about 1% (v/v) to about 3% (v/v), from about 1.5% (v/v) to about 2.5% (v/v), or about 2% (v/v). A particularly suitable polyoxyethylene surfactant is polysorbate 20 (Tween-20), which may present in the first lysis solution at, for example, a concentration of from about 1% (v/v) to about 10% (v/v), from about 2% (v/v) to about 8% (v/v), from about 2% (v/v) to about 6% (v/v), from about 3% (v/v) to about 5% (v/v), or about 4% (v/v); and polysorbate 20 may be used at an effective concentration of from about 0.5% (v/v) to about 5% (v/v), from about 1% (v/v) to about 4% (v/v), from about 1% (v/v) to about 3% (v/v), from about 1.5% (v/v) to about 2.5% (v/v), or about 2% (v/v). Other suitable detergents include, e.g., nonyl phenoxypolyethoxylethanol (NP-40), or a polyethylene oxide surfactant such as Triton X-100. In some embodiments of the method, the first solution includes at least two detergents. For example, in some variations in which the first solution comprises saponin, the lysis solution further contains polysorbate 20 as a second detergent.

In typical variations, the first solution further contains a buffer, which is typically present at a concentration sufficient to maintain a pH of, for example, from about 6.0 to about 10.0, from about 6.5 to about 9.0, from about 7.0 to about 8.0, or from about 7.2 to about 7.6. Suitable buffers include Tris (2-amino-2-(hydroxymethyl)-1,3-propanediol), PIPES (piperazine-N,N′-bis(2-ethanesulfonic acid)), HEPES (4-(2-hydroxyethyl)-1-piperazineethanesulfonic acid), phosphate, citrate, succinate, and histidine. In certain embodiments, the buffer is Tris, which may be present in the first solution at, for example, a concentration of from about 20 mM to about 200 mM, from about 30 mM to about 100 mM, from about 30 mM to about 50 mM, from about 35 mM to about 45 mM, or about 40.5 mM; and Tris may be used at an effective concentration (i.e., concentration in the first lysis reaction after mixing with the sample, or concentration in the final lysate) of from about 10 mM to about 100 mM, from about 15 mM to about 50 mM, from about 15 mM to about 25 mM, from about 18 mM to about 23 mM, or about 20.25 mM. Other suitable concentrations of buffers for formulations in accordance with the present disclosure can be readily determined by one of ordinary skill in the art.

The first lysis reagent and sample are admixed to induce sufficient lysis of the mammalian cells to allow the resulting first lysate to pass through the filter, while leaving microbial cells, if present, sufficiently intact to remain trapped on the filter surface. Preferably all or substantially all (e.g., at least 80%) of the microbial cells present in the sample remain intact following selective lysis of the mammalian cells. In some variations, at least 90%, at least 95%, at least 96%, at least 97%, at least 98%, or at least 99% of the microbial cells in the sample remain intact. In typical variations, a separate incubation time prior to passing the lysate through the filter is unnecessary and sufficient lysis is achieved upon mixing of the first lysis reagent and sample. In other embodiments, a short incubation time prior to passing the lysate through the filter may be used (for example, an incubation time of no more than 10 minutes, no more than five minutes, or no more than one minute). Exemplary temperature ranges for the first lysis reaction include 5-50° C., 10-45° C., 15-37° C., 20-30° C., 22-27° C., or 25° C. Ambient (room) temperature is particularly suitable.

In some variations comprising lysis of blood samples with the first lysis solution, the first lysis solution includes guanidine hydrochloride, saponin, and polysorbate 20, wherein (i) the effective concentration of guanidine hydrochloride in the first lysis reaction is at least about 1.5 M, at least 1.8 M, or at least about 2 M, (ii) the effective concentration of saponin in the first lysis reaction is at least about 1.5% (w/v), at least about 1.8% (w/v), or at least about 2% (w/v), and (iii) the effective concentration of polysorbate 20 in the first lysis reaction is at least about 0.5% (v/v), at least about 1% (v/v), at least about 1.5% (v/v), or at least about 2% (v/v). Such variations are particularly suitable for lysis of blood samples with high leukocyte and thrombocyte count, inducing sufficient lysis to allow the resulting first lysate to pass through the filter (e.g., for lysis of laboratory-made buffy coat samples, which may have a normal leukocyte (WBC) count in the range of about 17,000-44,000/μl, a erythrocyte (RBC) count of about 4×10⁶/μl, and a thrombocyte (PLT) count in the range of about 1.2-2.1×10⁶/μl).

The first lysate obtained with the selective lysis of mammalian cells (step (b)) is passed through a filter having a pore size that retains the intact microbial cells while passing the lysed mammalian material, thereby separating the intact microbial cells from the lysed mammalian cells. Portions of the first lysate that flows through the filter, including the lysed mammalian cells, may be collected, e.g., in a waste chamber.

Filtration is performed using a filter having a suitable pore size to capture microbial cells of interest. The filter may have a pore size of about 1 μm or less, preferably a pore size of about 0.5 μm or less, and more preferably a pore size of 0.25 μm or less. In some variations, the filter has a pore size of from about 0.1 μm to about 1 μm, from about 0.1 μm to about 0.5 μm, from about 0.2 μm to about 1 μm, or from about 0.2 μm to about 0.5 μm. In some embodiments, the filter has a pore size of about 0.22 μm. Suitable filter materials include, for example, polyethersulfone (PES), cellulose, nylon, polyvinylidene fluoride (PVDF), polycarbonate, and glass fiber (e.g., borosilicate glass fiber).

In some embodiments, the filter comprises an asymmetric structure. The asymmetric structure provides a kind of “built-in” prefilter and larger effective membrane area: the larger pores on an upstream side of the membrane trap larger particles on top of the membrane, whereas smaller particles are able to continue downstream to the interior or through the membrane, according to their size. The pore size gradually gets smaller from the upstream side of the membrane to the downstream side of the membrane, thus trapping particles to different parts of the membrane according to their sizes. In contrast, track-edged membranes gather all particles larger than the pore size to the same level, clogging the membrane more easily. Asymmetric filters may have a pore size from about 5 μm to about 20 μm on a first side of the asymmetric filter and a pore size of up to about 0.2 μm on a second side of the asymmetric filter.

Following the filtration step, the filter may, optionally, be washed to remove residual first solution and mammalian cell debris before step (d) (i.e., before contacting the filter containing retained microbial cells with the second solution). Washing is typically performed by passing a wash buffer through the filter. In some embodiments, the step (c) (i.e., filtering the first lysate through the filter) and the wash step are performed in multiple, successive iterations before step (d), wherein each iteration of step (c) comprises filtering a portion of the first lysate through the filter and each iteration of step (c) is followed by passing the wash buffer through the filter.

Suitable wash buffers may include a detergent and may further include a chelating agent (e.g., 2,2′,2″,2′″-(Ethane-1,2-diyldinitrilo)tetraacetic acid (EDTA)). For example, in some variations, a wash buffer includes a non-ionic detergent such as, e.g., polysorbate 20; in some such embodiments, the wash buffer further includes Tris and/or EDTA. Suitable concentrations of polysorbate 20 in a wash buffer include concentrations of from about 0.5% (v/v) to about 5% (v/v). In other variations, a wash buffer does not contain a detergent. For example, in some such variations, the wash buffer contains sodium hydroxide, dimethyl sulfoxide (DMSO), glycerol, a chelating agent (e.g., EDTA), and a buffer (e.g., Tris). In more particular variations of a wash buffer without a detergent, the wash buffer is the same as the second solution as described herein or is a diluted concentration of the wash buffer. In yet other embodiments, a wash buffer is a buffered solution selected from TE buffer (i.e., a buffer containing Tris and EDTA), Tris buffered saline (TBS), and phosphate buffered saline (PBS).

Following filtration of the first lysate and optional washing of the filter, the filter containing the retained, intact microbial cells is contacted and incubated with the second solution under conditions sufficient to facilitate release of the microbial cells from the filter. In some preferred variations, the second lysis solution comprises sodium hydroxide, a chelating agent, and a buffer. In some such embodiments, sodium hydroxide is present in the second lysis solution at a concentration of from about 10 mM to about 100 mM, from about 10 mM to about 80 mM, from about 20 mM to about 60 mM, from about 20 mM to about 50 mM, from about 20 mM to about 40 mM, or about 30 mM; and/or the chelating agent is EDTA and is present in the second lysis solution at a concentration of from about 0.1 mM to about 4 mM, from about 0.25 mM to about 3 mM, from about 0.5 mM to about 2 mM, or about 1 mM. Suitable buffers for use in the second lysis solution may include buffers as previously described for the first solution; for example, in some variations, the buffer is Tris and is present in the second lysis solution at a concentration of from about 1 mM to about 50 mM, from about 2 mM to about 25 mM, from about 5 mM to about 10 mM, or about 7.5 mM. In some embodiments, a second solution (e.g., a second lysis solution comprising sodium hydroxide and a chelating agent as described above) contains dimethyl sulfoxide (DMSO) (e.g., at a concentration of from about 2% (v/v) to about 20% (v/v), from about 2% (v/v) to about 15% (v/v), from about 5% (v/v) to about 20% (v/v), from about 5% (v/v) to about 15% (v/v), or about 10% (v/v)) and/or glycerol (e.g., at a concentration of from about 1% (v/v) to about 15% (v/v), from about 2% (v/v) to about 12% (v/v), from about 5% (v/v) to about 10% (v/v), or about 7% (v/v)).

The actions of the second solution in step (d) are not necessarily limited only to good release of the filter content but may also, e.g., dissolve possible micro/macro-coagulants formed in a sample. For example, the content of a blood sample can vary according to a person and the patient's condition, and chemical treatment with a second solution may be used to ensure that no interfering agglutinates or coagulants are present in downstream processes.

In some variations of a method as above, the conditions that facilitate release of the microbial cells include incubating the filter with the second solution at a temperature of, for example, from about 70° C. to about 99° C., from about 80° C. to about 99° C., from about 80° C. to about 95° C., or from about 90° C. to about 95° C. The filter may be heated in the presence of the second solution using an externally-controlled heat source embedded in or in contact with the filter or induction-mediated heating, where electrically-conductive or semi-conductive material like brass, copper, steel, iron, aluminum, graphite, carbon, or silicon is embedded on/in the filter matrix by, e.g., lamination of a porous mesh or co-molding with the actual filter material. In typical variations that use an external heating element, the heating element is located on the same side of the filter initially contacted by the first lysate at step (c) (also referred to herein as the “inlet side”). Exemplary times for incubating the filter with the second solution, optionally in the presence of heat, include 1-30 minutes, 2-20 minutes, 3-15 minutes, 4-12 minutes, 5-10 minutes, or 5-8 minutes.

Following release of the microbial cells, released microbial cells are collected from the filter at step (f). In this step, the filter content is not collected through the filter membrane (i.e., via “co-directional elution”) but is instead collected from the same side initially contacted by the first lysate, i.e., using a “reverse-flow” collection method. The use of an asymmetric filter in certain embodiments is particularly amenable to reverse-flow collection of the filter content since the content—packed inside the small membrane pores and stuck by the decreasing pore size—can be released from the side of the filter with larger pore size. In addition, with reverse-flow collection, the content of most interest (the content from the side initially contacted by the first lysate, i.e., the “inlet” side) is released from the filter first. Reverse-flow elution may increase downstream assay sensitivity in certain embodiments.

In some embodiments, microbial cells are collected using an elution buffer to elute the microbial cells from the filter, wherein the direction of fluid flow through the filter is opposite the direction of fluid flow applied during the filtering step (c). Suitable elution buffers may include a detergent and may further include a chelating agent (e.g., EDTA). For example, in some variations, an elution buffer includes a non-ionic detergent such as, e.g., polysorbate 20; in some such embodiments, the elution buffer further includes Tris and/or EDTA. Polysorbate 20 may be present in an elution buffer at, e.g., a concentration of from about 0.5% (v/v) to about 5% (v/v). In other variations, an elution buffer does not contain a detergent. For example, in some such variations, the elution buffer contains sodium hydroxide, dimethyl sulfoxide (DMSO), glycerol, a chelating agent (e.g., EDTA), and a buffer (e.g., Tris). In more particular variations of an elution buffer without a detergent, the elution buffer is the same as the second solution as described herein or is a diluted concentration of the elution buffer. In yet other embodiments, an elution buffer is a buffered solution selected from TE buffer (i.e., a buffer containing Tris and EDTA), Tris buffered saline (TBS), and phosphate buffered saline (PBS). The velocity of fluid flow in the reverse flow elution step may be, for example, up to about 20 ml/min (e.g., from about 10 ml/min to about 20 ml/min, or about 15 ml/min).

In alternative variations, microbial cells are collected by aspirating the microbial cells from the filter. In such embodiments, the microbial cells are aspirated from the same side of the filter initially contacted by the first lysate at step (c). Aspiration is particularly advantageous for facilitating enrichment of the microbial cell content since this collection method can be achieved without any elution buffer and therefore without any dilution. Aspiration can also be easier to implement in automated systems.

Following collection of the microbial cells from the filter, the cells are lysed off-filter to obtain a second lysate containing microbial cell analytes released from the intact organisms. The collected microbial cells may be lysed by a chemical, enzymatic, and/or physical lysis method. For example, lysis of the microbial cells may comprise (a) chemical lysis using a detergent, a chaotropic salt, an alcohol (e.g., glycerol), and/or an extreme pH value; (b) enzymatic lysis using an enzyme that is effective to degrade one or more microbial cell wall components (e.g., peptidoglycan, chitin, proteins) (e.g., an enzyme selected from mutanolysin, lyticase, lysozyme, endoglucanase, protease, chitinase, and combinations thereof); (c) physical lysis using heat; (d) physical lysis with direct electricity (electrolysis); (e) sonication/ultrasound with a sonotrode; (f) physical lysis using bead-beating (e.g., using a MagNA Lyser bead-beating instrument); (g) physical lysis using radiolysis; or (h) any combination of chemical, enzymatic, and/or physical lysis. In some variations, the microbial cells are lysed using electromagnetic lysis.

Following microbial cell lysis, the second lysate may be neutralized by adding a suitable neutralizing buffer. For example, in certain embodiments comprising the use of sodium hydroxide in the second solution and/or elution buffer as described herein, a neutralizing buffer contains hydrochloric acid and Tris (e.g., a 20× neutralizing buffer comprising HCl at a concentration of about 500 mM and Tris at a concentration of about 50 mM). In other embodiments, neutralization is not done separately but is instead achieved during subsequent processing of the sample for analysis of the isolated target analyte. For example, in certain embodiments comprising analyzing steps that include an in vitro nucleic acid amplification reaction (see, e.g., discussion of target nucleic acid amplification, infra), an amplification (e.g., PCR) reaction mixture is buffered so that the mixture of amplification reaction components and eluate is less alkaline than can be tolerated in the final reaction mixture.

A method for isolating a microbial cell analyte as above may further include analyzing the isolated analyte. The type of assay depends on the analyte.

For example, in some variations wherein the analyte is a nucleic acid, analyzing the isolated nucleic acid includes amplification. In such embodiments, the isolated nucleic acid analyte is used as a template in an in vitro nucleic acid amplification reaction, utilizing at least two amplification oligomers flanking a target sequence within the nucleic acid analyte, to generate an amplification product corresponding to the target sequence. A nucleic acid analyte can be amplified using methods such as isothermal amplification reactions (e.g., transcription mediated amplification (TMA), nucleic acid sequence-based amplification (NASBA), loop mediated isothermal amplification (LAMP), polymerase spiral reaction (PSR) (Liu et al., Sci. Rep. 5:12723, 2015), ligase chain reaction (LCR), and other isothermal amplification methods), or temperature cycling amplification reactions (e.g., polymerase chain reaction (PCR) or other temperature cycling amplification methods), or other amplification methods. In particular embodiments comprising PCR, the PCR is selected from quantitative PCR (qPCR) and real time PCR (rt-PCR).

Amplification may be performed with or without prior capture of the nucleic acid analyte. In some variations comprising a capture step, the isolated nucleic acid analyte is captured by hybridizing the nucleic acid to an immobilized capture probe attached to a solid support prior to the amplification step.

Detection of the amplified nucleic acid analyte products can be performed during amplification (real-time) or following amplification (end-point) by using any known method. Amplified nucleic acids may be detected in solution phase or by immobilizing them on a solid support (e.g., a nucleic acid array) and detecting labels associated with them (e.g., an intercalating agent such as ethidium bromide). Some detection methods use detection probes complementary to a sequence in the amplified product and detect the presence of the probe:product complex (e.g., by detecting a label joined to the probe), or use a complex of probes to amplify the signal detected from amplified products (see, e.g., U.S. Pat. Nos. 5,424,413, 5,451,503 and 5,849,481). Other detection methods use a probe in which signal production is linked to the presence of the target sequence because a change in signal results only when the labeled probe binds to amplified product, such as in a molecular beacon, molecular torch, or hybridization switch probe (e.g., U.S. Pat. Nos. 5,118,801, 5,210,015, 5,312,728, 5,538,848, 5,541,308, 5,656,207, 5,658,737, 5,925,517, 6,150,097, 6,361,945, 6,534,274, 6,835,542, and 6,849,412; and U.S. Pub. No. 2006/0194240 A1). Such probes typically use a label (e.g., fluorophore) attached to one end of the probe and an interacting compound (e.g., quencher) attached to another location of the probe to inhibit signal production from the label when the probe is in one conformation (“closed”) that indicates it is not hybridized to amplified product, but a detectable signal is produced when the probe is hybridized to the amplified product which changes its conformation (to “open”). Detection of a signal from directly or indirectly labeled probes that specifically associate with the amplified product indicates the presence of the target nucleic acid that was amplified.

In particular variations, an amplification and detection assay for analyzing isolated nucleic acid is a qPCR assay. Such assays include forward and reverse primers for target amplification, and a target-specific detection probe labeled with a fluorophore at the 5′-end and a quencher at the 3′-end (also referred to as a hydrolysis probe or TaqMan probe). In this format, the quencher disables the fluorophore emission. If the target nucleic acid is present in the sample, the probe binds to the complementary sequence within the amplification target region. As the polymerase extends the 3′-end of the primer and synthesizes a nascent complementary strand, the 5′ to 3′ exonuclease activity of the polymerase degrades the bound probe, thus disabling the quencher's effect on fluorophore. This is seen in increasing fluorescence intensity, according to amplification rate.

The following examples are provided to illustrate certain disclosed embodiments and are not to be construed as limiting the scope of this disclosure in any way.

### EXAMPLE 1

A protocol for microbial cell analyte enrichment from a larger sample volume containing mammalian cells was conducted manually as described below.
1) Take 4 ml of donor blood sample to 50 ml falcon-tube.
2) Add microbes under study to 5 cfu/ml concentration to the blood sample.
3) Add 4 ml of hemolysis solution (4M guanidine hydrochloride, 4% (v/v) Tween 20, 4% (w/v) saponin, 40.5 mM Tris) to the blood sample and mix by pipetting up-and-down for 20 times (this for selective lysis, i.e., to lyse the human cells).
4) Take the hemolyzed blood (now 8 ml) to 10 ml syringe.
5) Connect the syringe to a syringe filter (perpendicularly) (25 mm filter in diameter, PES membrane, 0.2/0.22 μm pore size).
6) Filter the hemolyzed blood through the filter (intact microbes are captured by the size-selective membrane, whereas majority of the human cell debris as well as human DNA are going through).
7) Detach the empty blood syringe from the filter.
8) Attach 20 ml syringe with 15 ml of wash solution (“GENTD”; 30 mM sodium hydroxide, 10% (v/v) DMSO, 7% (v/v) glycerol, 1 mM EDTA, 7.5 mM Tris) to the filter.
9) Infuse the wash solution through.
10) Cap the inlet and outlet of the filter (the filter contains now also GENTD), turn the filter and place it on top of the heater set to 95° C., inlet downwards (membrane-captured material with intact microbes are in the inlet-side of the membrane, thus heating is more efficient when provided to the inlet-side). Incubate 15 min.
11) OPTION A:
   a. After incubation, and letting cool down for 3 minutes, remove the caps and attach the filter outlet to a 3-way connector, connected to a TE-buffer filled syringe in infusion pump.
   b. Attach an empty 1 ml syringe to the filter inlet and make automated infusion with 350 μl of TE-buffer at 15 ml/min speed (=physical cleaning of the filter, i.e., removal of the filter-captured content with microbes).
   c. Detach the 1 ml syringe with the eluate (containing the filter content with intact microbes/microbial analytes), add it to MagNA Lyser bead beating tube and do mechanical lysis.
OPTION B:
   d. After incubation, and letting cool down for 3 minutes, remove the caps and attach the filter inlet to an empty 20 ml syringe.
   e. Aspirate the filter-content to the syringe (against the vacuum, as the membrane does not let air through).
   f. Detach the syringe (by actively holding the plunger in the 20 ml syringe against the vacuum) and transfer the eluate to a preferred tube for further processing with lysis method (MagNA Lyser bead beating).
12) Analyze the bead-beated eluate in PCR.

In this protocol, two steps allow microbes to be collected from the filter: chemical cleaning (GENTD incubation, step 10) and physical cleaning (reverse-flow, step 11).

Following step 9, the captured content of blood debris (which was not able to be removed by the wash step) with the intact microbes is packed against the membrane and inside the membrane pores. To release the captured content from the membrane in the physical cleaning step (step 11), the membrane is incubated with GENTD. Heating speeds up the reactions made by GENTD (i.e., denaturation and dissolution of the content). The chemical incubation likely lyses certain type of microbes (affected by the heating and the reagents in GENTD), but the tough-to-lyse Gram-positive microbes and yeasts stay intact. Lysis inside the filter is not needed but doesn't matter either, as the reverse-flow elution/aspiration step collects the content of interest from the filter (including possibly freed DNA).

In step 11, Option A, TE buffer was used to push the content of the filter out in a reverse-flow elution step. In this otherwise manual protocol, this step was performed with an infusion pump to the know the speed for physical cleaning (15 ml/min was tested and shown to be efficient for the physical cleaning step). The volume inside the filter is approximately 250 μl, meaning that there was 250 μl of GENTD inside the filter during the incubation step. When pushing 350 μl of liquid to the filter, approximately 250 μl came out (this was due to the quite large inlet and outlet parts of the commercial filter, which take part of the elution liquid). The elution liquid (which can also be another buffer or even GENTD) did not push the content completely out as it mixed with it. But the content from inlet side comes out first, and the captured material is in the inlet side, so the content of interest comes out first. According to the results, this protocol collected well even at low concentrations of analytes, meaning that the most concentrated part of the content was within that 250 μl eluate volume.

An alternative collection step 11, Option B, was also used. To replace reverse-flow elution, aspiration was used to collect the filter-content. By using a large syringe (10 or 20 ml), the plunger's withdrawal force was capable of pulling a similar volume from the filter as was eluted out by the push/reverse-flow step, without any dilution as no elution buffer was used. As with reverse-flow elution, the content of most interest (the content from inlet side) comes out first using the alternative aspiration step.

### EXAMPLE 2

A microbial cell enrichment protocol similar to the method described in Example 1 but utilizing multiple iterations of the selective filtering step followed by a wash (rinsing) step was conducted manually. This protocol is summarized below.
1) 4 ml of blood sample is mixed with 4 ml of hemolysis solution (same as Example 1).
2) Filtering is performed using a syringe filter (syringe filter is the same as in Example 1):
   a. First “priming” the filter with 2 mL rinsing solution (TE+2% (v/v) Tween-20);
   b. 2 ml of hemolyzed blood is filtered;
   c. 2.5 ml of rinsing solution;
   d. 2 ml of hemolyzed blood;
   e. 2.5 ml of rinsing solution;
   f. 2 ml of hemolyzed blood;
   g. 2.5 ml of rinsing solution;
   h. 2 ml of hemolyzed blood;
   i. 2.5 ml of rinsing solution;
3) 10 ml of Wash solution (GENTD; see Example 1) is filtered through.
4) Filter incubation by external heating (90° C., 15 minutes).
5) Aspiration to collect the filter content (see Example 1).
6) Mechanical lysis using MagNA Lyser bead beating.
7) Analyze the bead-beated eluate in PCR.

### EXAMPLE 3

This example describes isolation and detection of microbial cell analytes from samples with low microbe concentration using a microbial cell enrichment protocol similar to the method described in Example 2. This study contained the following:
1) 5 CFU/ml spiked samples (23 different microbes or targets);
2) Multiple rinsing/washing steps during hemolyzed blood filtering (TE+2% Tween-20: 1, 2.5, 2.5, 2.5, 3.5 ml);
3) Incubation with GENTD w/ 30 mM NaOH, 10% DMSO, 7% Glycerol;
4) Aspiration;
5) Addition of 0.5% foam-BAN to the aspirate to prevent foaming during lysis;
6) MagNA Lyser bead beating for lysis;
7) Neutralization;
8) 12 replicates on PCR plate;
9) 50 CFU/ml positive control (Made by spiking microbes directly to GENTD w/60 mM NaOH 10% DMSO, 7% Glycerol, lysed with MagNA Lyser, neutralized and analyzed in PCR; represents the concentration which should be achieved when doing enrichment for 5 CFU/ml spiked samples);
10) Plating the microbe stocks used for spiking to check the actual used cell concentration.

Table 1 below shows the microbial strains tested and the actual spiked cell concentrations according to plate counts (goal 5 CFU/ml).

TABLE 1

| | Spike conc. |
|---|---|
| Microbial strain | (CFU/ml) |
| Escherichia coli (K1) | 8 |
| Haemophilus influenzae | 19 |
| Neisseria meningitidis | 11 |
| Salmonella enterica | 8 |
| Acinetobacter baumannii (OXA- | 10 |
| 51) | |
| Stenotrophomonas maltophilia | 3 |
| Pseudomonas aeruginosa | 11 |
| Klebsiella pneumoniae | 4 |
| Enterobacter cloacae (KPC) | 8 |
| Staphylococcus aureus | 10 |
| Staphylococcus epidermidis | 3 |
| Staphylococcus lugdunensis | 12 |
| Streptococcus pneumoniae | 5 |
| Streptococcus agalactiae | 7 |
| Streptococcus pyogenes | 8 |
| Enterococcus faecium | 6 |
| Enterococcus faecalis | 9 |
| Listeria monocytogenes | 7 |
| Clostridium difficile | N/A |
| Cryptococcus neoformans, ATCC | 18 |
| MYA-4567, non-induced | |
| Cryptococcus neoformans, ATCC | 13 |
| MYA-4567, induced | |
| Cryptococcus neoformans, ATCC | 11 |
| 208821, non-induced | |
| Cryptococcus neoformans, ATCC | 4 |
| 208821, induced | |
| Candida albicans | 13 |
| Candida krusei | 6 |

As it was known that Cryptococcus is a challenging target pathogen to get lysed, the tendency of most cryptococcal species to form a polysaccharide capsule as a response to environmental stimulation was also considered. The capsule is the main virulence factor for these species, and the cells having the capsule are bigger in size. In this study, two different strains of C. neoformans were tested, as the strain ATCC 208821 is known to form the capsules more easily than the strain ATCC MYA-4567. Capsule-induced and non-induced C. neoformans cells were tested and the results were compared. The induction protocol was as follows: cryptococcal cells grown over night on sabouraud dextrose agar plate at 30° C. in aerobic conditions, were transferred into broth containing DMEM+2 mM MgCl₂+10% FBS, and incubated for 24-48 h at 37° C. with 10% CO₂. After incubation, the formation of polysaccharide capsules was observed under a microscope using Nigrosin staining.

The eluates were analyzed by Biorad CFX PCR using in-house PCR assays.

The results of this study are shown in Tables 2 and 3 below.

TABLE 2
PCR Analysis of Enriched sample vs Positive
Control Having the Concentration Goaled in Enrichment

| | Enrichment | Pos ctrl |
|---|---|---|
| Microbial Strain | (Cq) | (Cq) |
| E. coli (K1) | 37.5 | 38.1 |
| N. meningitidis | 38.8 | 40.5 |
| S. aureus | 38.3 | 38.7 |
| C. albicans | 37.8 | 39.7 |
| S. agalactiae | 37.8 | 36.9 |
| P. aeruginosa | 36.2 | 37.5 |
| H. influenzae | 37.8 | 37.5 |
| S. pneumoniae | 38.7 | 37.7 |
| S. enterica | 38 | 38.7 |
| A. baumannii | 38.6 | 38.8 |
| L. monocytogenes | 39 | 38.8 |
| E. faecalis | 38 | 37.9 |
| E. faecium | 38.8 | 39 |
| K. pneumoniae (VIM) | 37.8 | 38.1 |
| E. cloacae (KPC) | 38.4 | 39.4 |
| S. epidermidis | 37.7 | 38.1 |
| S. lugdunensis | 38.2 | 38.1 |
| S. maltophilia | 38.6 | 39.2 |
| C. difficile | 38.8 | 37.2 |
| S. pyogenes | 38.5 | 38.3 |
| C. krusei | 32.1 | 32.1 |
| C. neoformans (Fa248, ind) | 37 | 38 |
| C. neoformans (Fa251, non-ind) | 37.3 | 37.6 |
| C. neoformans (Fa251, non-ind) | 37.4 | 38.6 |
| C. neoformans (Fa251, ind) | 38.1 | 39.3 |
| C. neoformans (Fa248, non-ind) | 38.4 | 38.3 |
| C. neoformans (Fa248, ind) | 38.3 | 39.1 |

TABLE 3
PCR Positivity Rate of 12 PCR Replicates

| | Enrichment | Pos ctrl |
|---|---|---|
| Microbial Strain | (pos repl.) | (pos repl.) |
| E. coli (K1) | 83 | 83 |
| N. meningitidis | 92 | 83 |
| S. aureus | 83 | 100 |
| C. albicans | 100 | 62 |
| S. agalactiae | 83 | 83 |
| P. aeruginosa | 100 | 92 |
| H. influenzae | 100 | 100 |
| S. pneumoniae | 58 | 100 |
| S. enterica | 83 | 75 |
| A. baumannii | 83 | 83 |
| L. monocytogenes | 83 | 83 |
| E. faecalis | 100 | 100 |
| E. faecium | 83 | 83 |
| K. pneumoniae (VIM) | 100 | 75 |
| E. cloacae (KPC) | 100 | 100 |
| S. epidermidis | 100 | 92 |
| S. lugdunensis | 100 | 100 |
| S. maltophilia | 100 | 83 |
| C. difficile | 100 | 100 |
| S. pyogenes | 100 | 75 |
| C. krusei | 100 | 100 |
| C. neoformans (Fa248, ind) | 50 | 8 |
| C. neoformans (Fa251, non-ind) | 100 | 56 |
| C. neoformans (Fa251, non-ind) | 89 | 58 |
| C. neoformans (Fa251, ind) | 33 | 8 |
| C. neoformans (Fa248, non-ind) | 83 | 70 |
| C. neoformans (Fa248, ind) | 75 | 20 |

Overall, the results show that the developed protocol is capable of enriching the tested microbes efficiently. A few of the microbes showed challenges in culturing and stock preparation, as discussed below.

S. pneumoniae: The enrichment protocol for this microbe was repeated several times, as the PCR positivity rates in the enriched samples were low. However, the positive controls prepared by beat beating in GENTD solution gave 100% positivity. Pneumococci has an autolysis tendency which is induced when the microbe growth reaches a stationary phase. Modelling of actual physiological growth conditions can be challenging for this microbe, and the actual protocol performance can be only studied when positive clinical blood samples are available.

C. difficile: C. difficile also has a tendency to start destroying its own DNA. Still, feasible detection was gained in the study. The cell counts on plates were not reliable (resulting only in 0-2 CFUs out of 100 CFU), most likely due to non-optimal plating conditions (not possible fully anaerobically).

C. neoformans: Some differences were seen in detection of capsule-induced and non-induced cells, indicating variance in lysis efficiencies, but still the positivity rates were similar or even better than the detectivity with positive control. The capsule structure changes the size of the microbe making the stock preparation difficult, but with all tested microbes, the plate counting showed the real spike concentration. The spike concentration according to plate count for the induced strain ATCC 208821 with the highest number of induced capsules, was 4 CFU/ml. With this low cell concentration and with partially capsuled cells, the detection rate was still 75% with average Cq value of 38.3.

Conclusions: All the tested target microbes can be detected with an at least acceptable positivity rate using the microbial cell enrichment protocol, with many of the tested microbial targets showing high positivity rates at or near 100%.

From the foregoing, it will be appreciated that, although specific embodiments of the invention have been described herein for purposes of illustration, various modifications may be made without deviating from the spirit and scope of the invention. All publications, patents, and patent applications cited herein are hereby incorporated by reference in their entireties for all purposes.

1. A method for selectively isolating a microbial cell analyte from a sample, the method comprising:
(a) providing a sample containing mammalian cells and potentially containing microbial cells;
(b) mixing the sample with a first solution that selectively lyses the mammalian cells to obtain a first lysate containing lysed mammalian cells and intact microbial cells, if present, wherein the first solution is a buffered solution comprising a chaotropic salt that is guanidine hydrochloride, and a detergent that is saponin;
(c) filtering the first lysate through a filter having a pore size that retains the intact microbial cells, wherein the pore size from about 0.1 μm to about 1 μm;
(d) contacting the filter containing the retained microbial cells with a second solution that is effective to allow release of the microbial cells from the filter, wherein the second solution is a buffered solution comprising sodium hydroxide at a concentration of from about 10 mM to about 100 mM, dimethyl sulfoxide (DMSO) at a concentration of from about 10% (v/v) to about 20% (v/v), glycerol at a concentration of from about 1% (v/v) to about 15% (v/v), and a chelating agent;
(e) incubating the filter with the second solution at an incubation temperature of from about 70° C. to about 99° C. to facilitate the release of the microbial cells from the filter;
(f) collecting the microbial cells from the filter, wherein said collection step comprises
(i) using an elution buffer to elute the microbial cells from the filter, wherein the direction of fluid flow through the filter is opposite the direction of fluid flow applied in the filtering step (c); or
(ii) aspirating the microbial cells from the filter, wherein the microbial cells are aspirated from the same side of the filter initially contacted by the first lysate at step (c); and
(g) lysing the microbial cells collected at step (f) to obtain a second lysate comprising an analyte released from the microbial cells, thereby selectively isolating a microbial cell analyte from the sample.

2. The method of claim 1, wherein the microbial cells are bacterial cells and/or yeast cells.

3. The method of claim 2, wherein the microbial cells are Gram-positive bacterial cells.

4. The method of claim 1, wherein the filter comprises polyethersulfone (PES), cellulose, nylon, polyvinylide fluoridene (PVDF), polycarbonate, or glass fiber.

5. The method of claim 1, wherein
the guanidine hydrochloride is present in the first solution at a concentration of from about 1 M to about 8 M.

6. The method of claim 1, wherein the first solution further comprises a second detergent that is polysorbate 20.

7. The method of claim 6, wherein the polysorbate 20 is present in the first solution at a concentration of from about 1% (v/v) to about 10% (v/v).

8. The method of claim 1, wherein during the mixing step (b) the ratio of the first lysis solution to the sample is about 1:1.

9. The method of claim 1, further comprising a washing step between steps (c) and (d), wherein the washing step comprises flowing a wash buffer through the filter.

10. The method of claim 9, wherein the filtering step (c) and the wash step are performed in multiple, successive iterations before step (d), wherein each iteration of step (c) comprises filtering a portion of the first lysate through the filter.

11. The method of claim 1, wherein lysing the microbial cells at step (g) comprises physical lysis.

12. The method of claim 11, wherein the physical lysis comprises electromagnetic lysis.

13. The method of claim 1, wherein the analyte is a nucleic acid.

14. The method of claim 1, further comprising analyzing the isolated analyte.

15. The method of claim 14, wherein analyzing the isolated nucleic acid comprises immobilizing the isolated nucleic acid or amplification product on a solid support.

16. The method of claim 1, wherein the filter comprises an asymmetric structure.

17. The method of claim 16, wherein pores on a first side of the asymmetric filter have a size from about 5 μm to about 20 μm and pores on a second side of the asymmetric filter have a size of up to about 0.2 μm.

18. The method of claim 1, wherein the saponin is present in the first solution at a concentration of from about 1% (w/v) to about 10% (w/v).

19. The method of claim 1, wherein the chelating agent in the second solution is 2,2′,2″,2′″-(Ethane-1,2-diyldinitrilo)tetraacetic acid (EDTA) and is present at a concentration of from about 0.1 mM to about 4 mM.

20. The method of claim 1, wherein the first solution is buffered with from about 20 mM to about 200 mM of a Tris buffer.

21. The method of claim 1, wherein the second solution is buffered with from about 1 mM to about 50 mM of a Tris buffer.

22. The method of claim 14, wherein the analyte is a nucleic acid, and wherein analyzing the isolated nucleic acid analyte comprises (i) performing a nucleic acid amplification reaction using the isolated nucleic acid as a template to generate an amplification product; and (ii) detecting the amplification product.
